(19) United States
(12) Patent Application Publication
(10) Pub. No.: US 2022/0040499 A1
(43) Pub. Date: Feb. 10, 2022

(54) BIODEGRADABLE ADHESIVE WITH RADIOISOTOPES

(71) Applicants: Charles Martin, III, Pepper Pike, OH (US); Vladimir Kepe, Shaker Heights, OH (US)

(72) Inventors: Charles Martin, III, Pepper Pike, OH (US); Vladimir Kepe, Shaker Heights, OH (US)

(21) Appl. No.: 17/398,118

(22) Filed: Aug. 10, 2021

Related U.S. Application Data
(60) Provisional application No. 63/063,752, filed on Aug. 10, 2020.

(51) Int. Cl. A61N 5/10; A61K 51/08; A61K 51/06; A61K 51/04

(52) U.S. Cl. CPC A61N 5/1007; A61K 51/08; A61N 2005/1096; A61K 51/0482; A61K 51/06

(57) ABSTRACT

A radioactive biodegradable adhesive system can include a first component and a second component. The second component can include a radioactive material. The first component and the second component are configured to polymerize to form a radioactive bolus. A method for treating a patient in need of brachytherapy can include providing the radioactive biodegradable adhesive system. The first component and the second component can be applied to a predetermined location to form the radioactive bolus at the predetermined location.

### CROSS-REFERENCE TO RELATED APPLICATIONS

This application claims the benefit of U.S. Provisional Application No. 63/063,752, filed on Aug. 10, 2020. The entire disclosure of the above application is incorporated herein by reference.

### FIELD

The present disclosure relates to the treatment of cancers and, more particularly, brachytherapy for cancer treatment.

### INTRODUCTION

This section provides background information related to the present disclosure which is not necessarily prior art.

Cancer is a major cause of death in the modern world. Effective treatment of cancer is most readily accomplished following early detection of malignant tumors. Most techniques used to treat cancer (other than chemotherapy) are directed against a defined tumor site in an organ, such as brain, breast, ovary, colon and the like.

When a mass of abnormal cells is consolidated and is sufficiently large, surgical removal, destruction of the tumor mass using heating, cooling, irradiative or chemical ablation becomes possible because the target is readily identifiable and localizable. However, it is not uncommon for a cancer that has initially occurred at a primary site to metastasize and spread into adjacent organs as diffuse clusters of abnormal cells or secondary tumors. Identification of primary and secondary tumors can permit localized treatment.

A number of techniques have been utilized or proposed to treat tumor growth, including radiation therapy, chemotherapy, and other treatment modalities. Radiation therapy, also referred to as radiotherapy, or therapeutic radiology, is the use of radiation sources in the treatment or relief of diseases. Radiotherapy typically makes use of ionizing radiation, deep tissue-penetrating rays, which can physically and chemically react with diseased cells to destroy them. Each therapy program has a radiation dosage defined by the type and amount of radiation for each treatment session, frequency of treatment session and total of number of sessions. Radiotherapy is particularly suitable for treating solid tumors, which have a well-defined spatial contour. Such tumors are encountered in breast, kidney and prostate cancer, as well as in secondary growths in the brain, lungs and liver.

To avoid adversely affecting any healthy region of the subject during radiotherapy, one attempts to maximize the radiation dosage administered to the target zone (to ensure killing the cancerous cells) while minimizing the dosage to other regions (to avoid undesirable damage). Most commonly, radiotherapy is used as an adjunct way of use, such as treating any remnant, not entirely removed, tumor cells by exposure to a radiation dose of an external source after the surgical opening of the human body, removal of the malignant tumor, and the suture of the body part(s). The radiation dose can also be applied directly to the remnant tumor cells before the suture of the body part(s) involved.

Brachytherapy, another form of radiation therapy, relies on implanting a radiation source in the body to provide localized treatment, as contrasted, for example, with treating a site from a distance by external beam radiation. To implant the radiation source, a predetermined treatment site can be accessed either percutaneously or via surgically/laparoscopically. By localizing the treatment to a specific area, nearby tissue can be less effected, while still delivering adequate radiation to destroy diseased cells.

Certain ways of applying brachytherapy are believed to undesirably cause side effects in patients due to the migration of the implanted radiation source or seed within the body of the patient. Examples undesirable side effects can include inadequate dosimetry and possible morbidity in distant organs. There is accordingly a continuing need for a localized radiation treatment system and method that militates against the migration of the radiation source during brachytherapy treatment.

### SUMMARY

In concordance with the instant disclosure, a localized radiation treatment system and method are provided that militate against the migration of the radiation source during brachytherapy treatment have been surprisingly discovered.

Certain embodiments of the present technology employ a radioactive biodegradable adhesive system that includes a radioactive polymerizing component and a first component. The radioactive polymerizing component can further include a radioisotope. The radioactive biodegradable adhesive system can be in the form of a precipitating hydrophobic compound. As further discussed herein, there are a plurality of approaches for constructing the radioactive biodegradable adhesive system.

In one embodiment, a radioactive biodegradable adhesive system can include a first component and a second component. The second component can include a radioactive material. The first component and the second component are configured to polymerize to form a radioactive bolus.

In another embodiment, a method for treating a patient in need of brachytherapy can include providing a radioactive biodegradable adhesive system. The system can include a first component and a second component. The second component can include a radioactive material. The first component and the second component are configured to polymerize to form a radioactive bolus. The first component and the second component can be applied to a predetermined location to form the radioactive bolus at the predetermined location.

In a non-limiting example, the non-radioactive polymerizing component can include one or more of polylactate and an ethylene vinyl alcohol copolymer. The radioactive biodegradable adhesive system can also be in the form of a precipitating hydrophobic compound. Advantageously, the protective nature of the precipitating hydrophobic compound is believed to slow down the nuclear half-life decay of the radioactive polymerizing component. Desirably, the increased nuclear half-life is believed to require a smaller dose of the radioactive polymerizing component to treat the cancer. A skilled artisan can select other suitable non-radioactive polymerizing components and radioactive biodegradable adhesive system formations, within the scope of the present disclosure.

The formation of the radioactive bolus according to the method and system of the present disclosure can mitigate against an opportunity for migration over time. Without being bound by any particular theory, it is believed that, as the radioactive bolus polymerizes in situ, it will intermesh mechanically with surrounding tissues, thereby anchoring the radioactive bolus to the surrounding tissues and minimizing subsequent movement away from the tissues over time.

In a particular embodiment, the radioactive biodegradable adhesive system can be formed through the polymerization of a monomer with a radioactive catalyst. The radioactive catalyst can include the radioisotope. In a non-limiting example, the radioactive catalyst can include yttrium-90 as the radioisotope. In a more specific example, the radioactive catalyst can include dodecane tetraacetic acid derivative. A skilled artisan can select other suitable radioisotopes and radioactive catalysts to form the radioactive biodegradable adhesive system, within the scope of the present disclosure.

In a particular example, the monomer can be specifically configured to present different properties based on a radical group of the monomer. In a more specific, non-limiting example, the radical group can include one of metacrylate (Me) and cyanoacrylate (CN). For example, with cyanoacrylate as the radical group of the monomer, the radioactive biodegradable adhesive system can present a strong, fasting acting adhesive feature. One skilled in the art can select other radical groups to present different properties, within the scope of the present disclosure.

In another embodiment, the radioactive biodegradable adhesive system can be constructed through crosslinking a first biopolymer with a second biopolymer. In one particular, non-limiting example, the first biopolymer acting as the non-radioactive polymerizing component can be chitosan, or otherwise known as poly-glucosamine. The first biopolymer can also be described as a non-radioactive crosslinker. In another specific non-limiting example, the second biopolymer can be a radioactive crosslinker. In an even more specific, non-limiting example, the radioactive crosslinker can include an aldehyde combined with yttrium-90 as the radioactive polymerizing component. It should be appreciated that crosslinkers other than aldehydes can be utilized to form the radioactive biodegradable adhesive system. Advantageously, the combination of a radioactive crosslinker with a non-radioactive crosslinker can provide sufficient polymerization while also providing an appropriate dose of localized radiation. A skilled artisan can select other suitable crosslinking biopolymers to form the radioactive biodegradable adhesive system, within the scope of the present disclosure.

In a particular embodiment, the radioactive biodegradable adhesive system can be constructed through crosslinking proteins with radioactive crosslinkers. As stated hereinabove, a non-limiting example of the radioactive crosslinker can include an aldehyde combined with yttrium-90 as the radioactive polymerizing component. As stated hereinabove, it should be appreciated that crosslinkers other than aldehydes can be utilized to construct the radioactive biodegradable adhesive system. Advantageously, any globular protein can be used as the non-radioactive polymerizing component.

In a specific embodiment, the radioactive biodegradable adhesive system can be constructed through crosslinking a radiolabeled peptide with a non-radioactive cross-linker. In a specific, non-limiting example, the radiolabeled peptide can be formed from a precursor such as a dodecane tetraacetic acid (DOTA) based chelator. One skilled in the art can select other radiolabeled peptides and suitable cross-linkers to construct the radioactive biodegradable adhesive system.

Further areas of applicability will become apparent from the description provided herein. The description and specific examples in this summary are intended for purposes of illustration only and are not intended to limit the scope of the present disclosure.

### DRAWINGS

This section provides background information related to the present disclosure which is not necessarily prior art.

FIG. 1 is a top plan view of a radioactive biodegradable system including an insertion device configured to dispense a first component and a second component to create a radioactive bolus of a radioactive biodegradable adhesive system, according to one embodiment of the present disclosure;

FIG. 2 is a cross-sectional view taken along the longitudinal axis of the insertion device at section line A-A in FIG. 1, depicting a first chamber and a second chamber configured to separately and simultaneously dispense the first component and the second component, further depicting arrows to indicate the flow of the components through the insertion device;

FIG. 2A is a cross-sectional side elevational view of the insertion tip of the insertion device, taken at callout 2A in FIG. 2, further depicting arrows to indicate the flow of the components through the insertion device;

FIG. 3 is a cross-sectional side-elevational view of the radioactive biodegradable system, depicting the first component and the second component contained within an insertion device of FIG. 1, further depicting the insertion device being percutaneously positioned to dispense and insert the non-radioactive polymerizing component and the radioactive polymerizing component at a predetermined mass for treatment;

FIG. 4 is a cross-sectional side-elevational view of the radioactive biodegradable adhesive system of FIG. 1, further depicting the inserted non-radioactive polymerizing component and the radioactive polymerizing component polymerizing into a radioactive bolus at a predetermined location within the body of the patient adjacent the predetermined mass for treatment;

FIG. 5 is a cross-sectional side-elevational view of the radioactive biodegradable adhesive system of FIG. 1, further depicting the inserted first component and second component polymerizing into a radioactive bolus at a predetermined location within the body of the patient and further within the predetermined mass for treatment, according to another embodiment of the present disclosure;

FIG. 6 is a cross-sectional side-elevational view of the radioactive biodegradable adhesive system of FIG. 1, further depicting the radioactive bolus applied over a surface which contains residual pieces of the predetermined mass for treatment, according to another embodiment of the present disclosure;

FIG. 7 is a cross-sectional side-elevational view of the radioactive biodegradable adhesive system of FIG. 1, further depicting the radioactive bolus brushed over a surface of a cavity where the predetermined mass for treatment has been resected, according to another embodiment of the present disclosure;

FIG. 8 is a schematic illustrating the radioactive biodegradable adhesive system from FIG. 1, depicting the polymerization of a monomer with a radioactive catalyst;

FIG. 9 is a schematic illustrating the radioactive biodegradable adhesive system from FIG. 4, depicting the radioactive catalyst being dodecane tetraacetic acid (DOTA) derivative, according to one embodiment of the present disclosure;

FIG. 10 is a schematic illustrating the radioactive biodegradable adhesive system from FIG. 1, depicting the crosslinking of a non-radioactive biopolymer crosslinker with a radioactive biopolymer crosslinker, according to another embodiment of the present disclosure;

FIG. 11 is a schematic illustrating the radioactive biodegradable adhesive system from FIG. 1, further depicting the crosslinking of a radioactive crosslinker with a protein, according to a further embodiment of the present disclosure;

FIG. 12 is a schematic illustrating the radioactive biodegradable adhesive system from FIG. 1, further depicting the crosslinking of a non-radioactive crosslinker with a radiolabeled peptide, according to one embodiment of the present disclosure; and

FIG. 13 is a flowchart depicting a method of for treating a patient in need of brachytherapy with the radioactive biodegradable adhesive system.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as can be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items can be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that can arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9,1-8,1-3,1-2,2-10,2-8,2-3,3-10,3-9, and so on.

As used herein, the term “bolus” is defined as meaning a body of polymerized material of any suitable size or shape, and which can include amorphous three-dimensional shapes, as well as layers or coatings of the polymerized material on internal surfaces within a body 107 of a patient. The bolus can include a selected radiation dose.

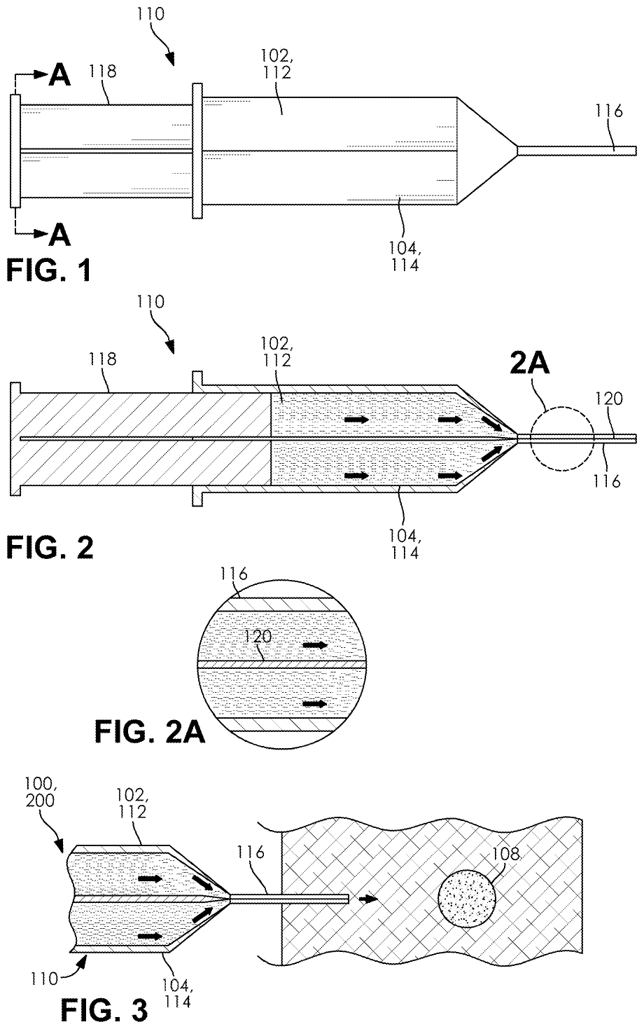

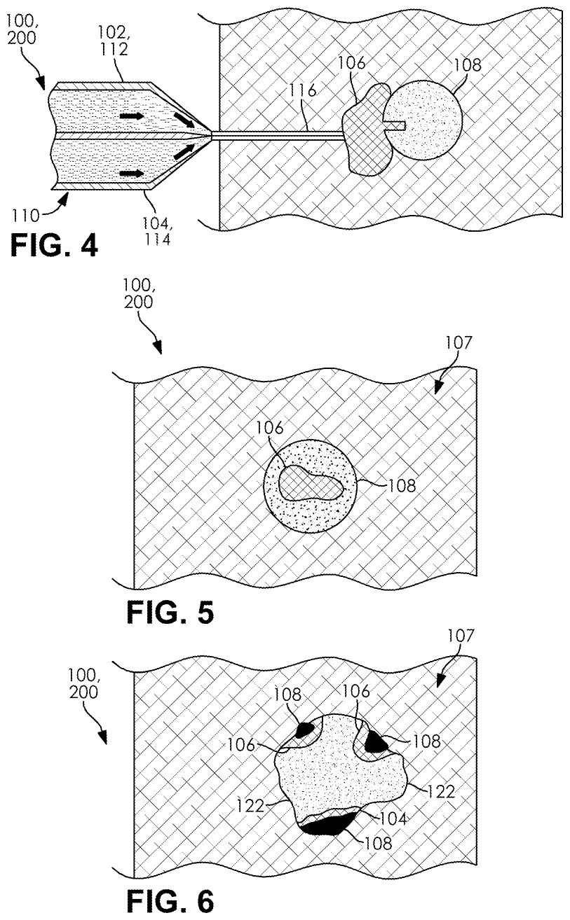

As shown in FIGS. 1-12, a radioactive biodegradable adhesive system 100 includes a first component 102 and a second component 104. The second component 104 can include a radioactive material. The first component 102 and the second component 104 can be configured to be mixed and polymerized. For example, the radioactive biodegradable adhesive system 100 can be polymerized according to reaction (1), where A represents the first component 102, B_{RAD} represents the second component 104, and (A−B_{RAD})ₓ represents x number of repeating polymerized units of the first component 102 and the second component 104:

A+B_{RAD}→(A−B_{RAD})ₓ (1)

It should be appreciated that the radioactive biodegradable adhesive system 100 can be configured to polymerize into a radioactive bolus 106. In particular, the radioactive biodegradable adhesive system 100 can be configured to be applied to a predetermined location within a body 107 of a patient adjacent a predetermined mass 108 for treatment. As a non-limiting example, the predetermined mass can be a tumor.

The formation of the radioactive bolus 106 of the present disclosure can mitigate against an opportunity for migration of the radioactive material through the body 107 of the patient over time. Without being bound by any particular theory, it is believed that, as the radioactive bolus 106 polymerizes in situ, it will intermesh mechanically with surrounding tissues, thereby anchoring the radioactive bolus 106 to the surrounding tissues and minimizing subsequent movement away from the tissues over time. Advantageously, the radioactive bolus 106 can deliver radioactive treatment directly to the effected location within the body 107 of the patient.

With reference to FIGS. 1-2, the radioactive biodegradable adhesive system 100 can include an insertion device 110. The insertion device 110 can include a first chamber 112, a second chamber 114, an insertion tip 116, and a force applicator 118. The first chamber 112 can contain the first component 102. The second chamber 114 can contain the second component 104. As non-limiting examples, the insertion tip can be a needle or a catheter having a central dividing wall 120. The dividing wall 120 can militate against an undesirable mixing of the components within the insertion tip 116, in operation. As a non-limiting example, the force applicator 118 can include a push rod in each of the chambers 112, 114. A skilled artisan can select other methods of dispensing the first component 102 and the second component 104, within the scope of the present disclosure.

The insertion device 110 can be configured to separately dispense both the first component 102 and the second component 104 simultaneously to the predetermined location within the body 107 of the patient. Advantageously, with the insertion device 110 separately and simultaneously inserting both the first component 102 and the second component 102, the radioactive biodegradable adhesive system 100 can be more accurately positioned at a desired location without undesirably polymerizing the first component 102 and the second component 104 during placement. Rather, the polymerization of the first component 102 and the second component 104 cannot occur until dispensed substantially at the predetermined location within the body 107 of the patient. More accurate placement of the first component 102 and the second component 104 can allow for use of less radioactive material to be disposed within the body 107 of the patient when treating the patient.

It should be appreciated that, according to certain embodiments, the insertion device 110 can be configured to separately dispense each of the first component 102 and the second component 104 out of a distal end of the insertion tip 116. It should be further appreciated that, in certain examples, there is no premixing of the components 102, 104. Rather, the components 102, 104 are not mixed or otherwise contact one another until the components 102, 104 are dispensed from the insertion device 110.

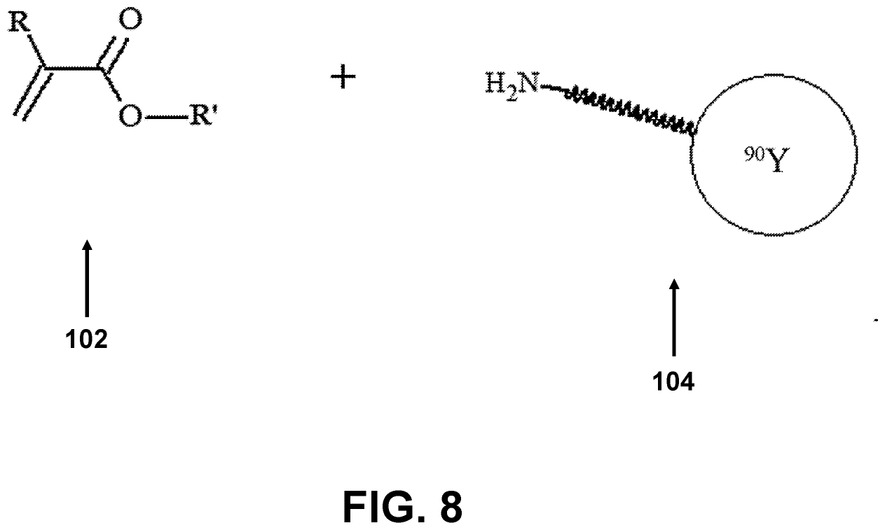

It should be appreciated certain considerations can be applied to the selection of each of the first component 102 and the second component 104. The components 102, 104 can be selected based upon a desired rate of polymerization, for example. It can be desirable in certain situations to select components 102, 104 that can polymerize faster than certain other components. It can also be desirable for the components to provide a slower rate of polymerization. The desired rate of polymerization can be selected based on the patient, including the location, size, and shape of the particular mass to be treated within the body 107 of the patient. The components 102, 104 can be selected based on an amount of free reactive groups. For example, as shown in FIGS. 8-12, the components can include reactive amine groups. Components 102, 104 having more free amine groups can polymerize faster than components 102, 104 with fewer free amine groups. A skilled artisan can select components 102, 104 with other suitable reactive groups, as desired.

In other examples, the components 102, 104 can be selected based on the type and location of the predetermined mass 108 within the body 107 of the patient. For example, the radioactive material of the second component 104 can include an alpha emitter or a beta emitter based on a treatment plan for the type of predetermined mass 108. For example, yttrium-90 can be utilized as the beta emitter. Each of the first component 102 and second component 104 can be selected based on the location of the predetermined mass 108. For example, the components 102, 104 that can be selected for a predetermined mass located in a bone of a patient can be different than the components 102, 104 that can be selected for a predetermined mass located in the soft tissue of a patient.

It should be appreciated that a predetermined amount of the radioactive material can be utilized within the radioactive biodegradable adhesive system 100. The predetermined amount of radioactive material can be provided by the second component 104. However, it should be appreciated that each unit of the second component 104 may not include radioactive material. Accordingly, the radioactive biodegradable adhesive system 100 can offer sufficient polymerization between the first component and the second component 104 while also allowing for control over the amount of radioactive material present in the final radioactive bolus 106. Desirably, the radioactive biodegradable adhesive system 100 also minimizes the time required to treat the predetermined mass 108. The predetermined mass 108 can be analyzed while a biopsy surgery is occurring. The dosage can then be calculated and immediately applied to the predetermined mass 108.

Certain embodiments of the second component 104 include where the second component is loaded with a selected dose of radiation by absorption, reaction, or chelation of radionuclide or radionuclide-containing compound. It is possible that not all sites of the second component 104 amenable to absorption, reaction, or chelation of the radionuclide or radionuclide-containing compound are occupied thereby. For example, a fraction of the second component 104 can be loaded as desired to achieve a selected radiation dose.

In a non-limiting example, the first component 102 can include polylactate and an ethylene vinyl alcohol copolymer. The polymerization reaction of the radioactive biodegradable adhesive system 100 can also be in the form of a precipitating hydrophobic compound. Advantageously, the protective nature of the precipitating hydrophobic compound is believed to slow down the nuclear half-life decay of the radioactive material of the second component 104. Desirably, the increased nuclear half-life is believed to require a smaller dose of the radioactive material of the second component 104 to treat the cancer. A skilled artisan can select other suitable components 102, 104 and radioactive biodegradable adhesive system 100 formations, within the scope of the present disclosure.

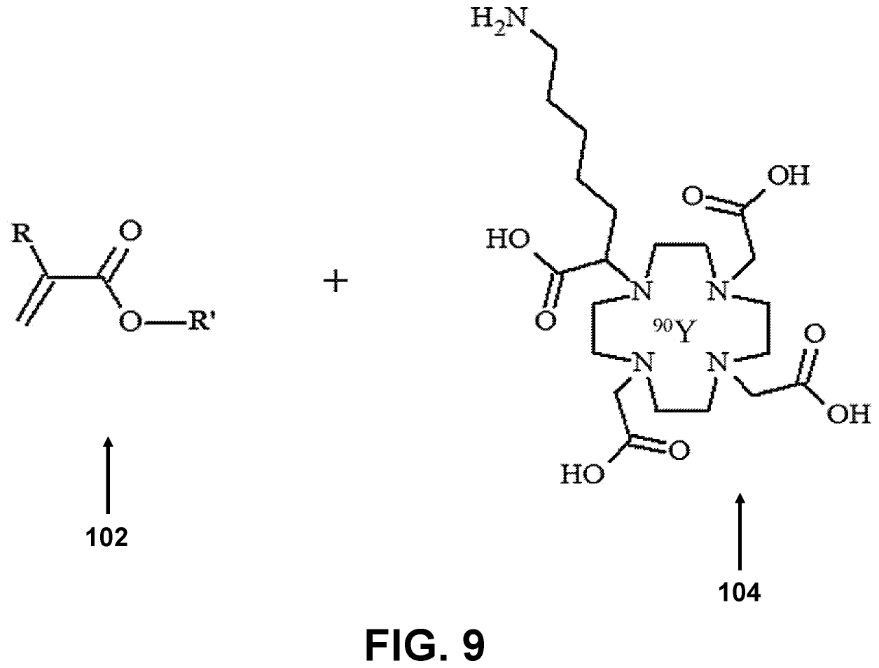

In an example embodiment, as shown in FIG. 8, the first component 102 can include a monomer. The second component 104 can include a radioactive catalyst. The radioactive catalyst can contain the radioactive material. Though yttrium-90 is shown throughout FIGS. 8-9 as the radioactive material of the second component 104, it should be appreciated that other alpha emitter and beta emitters can be utilized within the second component. In a more specific example, as shown in FIG. 9, the radioactive catalyst can be dodecane tetraacetic acid derivative. A skilled artisan can select other suitable radioactive catalysts to form the second component 102 of the radioactive biodegradable adhesive system 100, within the scope of the present disclosure.

In further reference to FIGS. 8-9, in a particular example, the monomer can be specifically configured to present different properties based on a radical group (represented as R′) of the monomer. In a more specific, non-limiting example, the radical group can be one of methacrylate and cyanoacrylate. For example, with cyanoacrylate as the radical group 120 of the monomer, the radioactive biodegradable adhesive system 100 can present a strong, fasting acting adhesive feature. One skilled in the art can select other radical groups to present different properties, within the scope of the present disclosure.

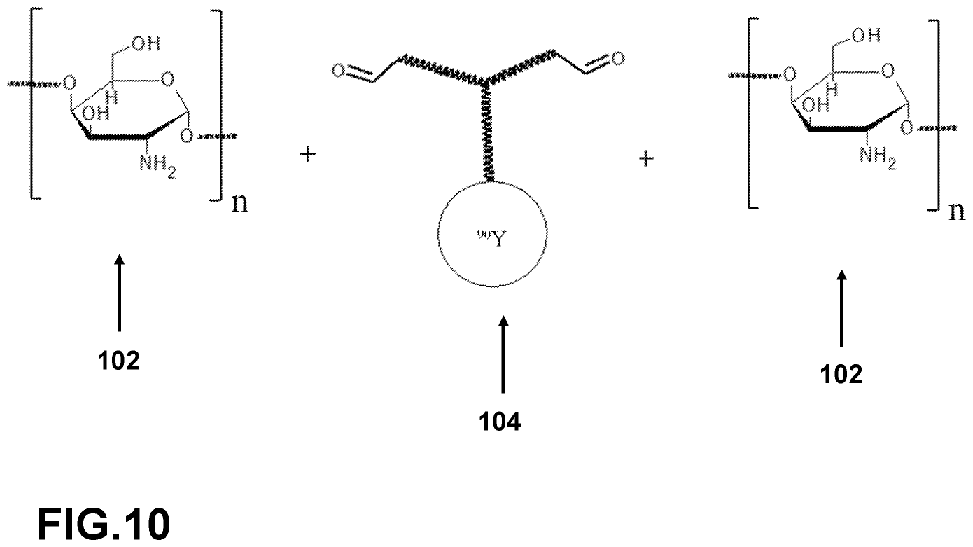

In another embodiment, as shown in FIG. 10, the components 102, 104 of the radioactive biodegradable adhesive system 100 can include biopolymers. In one particular, non-limiting example, the first component 102 can include chitosan, or otherwise known as poly-glucosamine. In another specific non-limiting example, the second component 104 include a radioactive crosslinker. The second component 104 can include an aldehyde combined with the radioactive material, such as yttrium-90. It should be appreciated that crosslinkers other than aldehydes can be utilized to form the radioactive bolus 106 of the radioactive biodegradable adhesive system 100. Advantageously, the combination of a radioactive crosslinker with a non-radioactive first component 102 can provide sufficient polymerization while also providing an appropriate dose of localized radiation. A skilled artisan can select other suitable crosslinking biopolymers to form the radioactive biodegradable adhesive system 100, within the scope of the present disclosure.

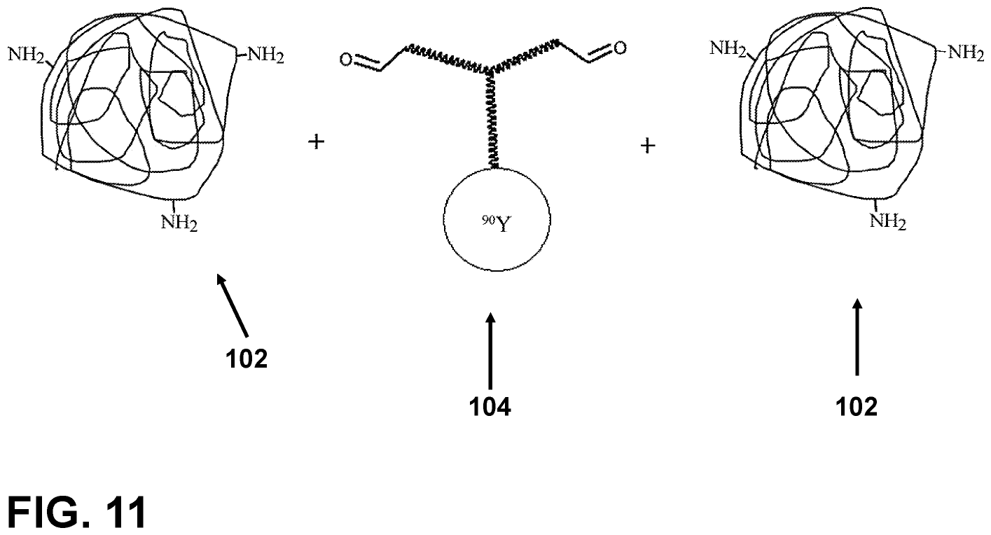

In a particular embodiment, as shown in FIG. 11, the first component 102 can be a protein. The protein can include reactive amine groups, including at the N-terminus of polypeptides and the side chains of certain amino acids (e.g., arginine, histidine, lysine, asparagine, glutamine, glycine). The second component 104 can include aldehydes combined with the radioactive material (shown herein as yttrium-90). The reactive amine groups and the aldehydes can react to allow for polymerization of the radioactive bolus. As stated hereinabove, it should be appreciated that crosslinkers other than aldehydes can be utilized to construct the radioactive biodegradable adhesive system 100. Advantageously, any suitable globular protein can be used as the first component 102.

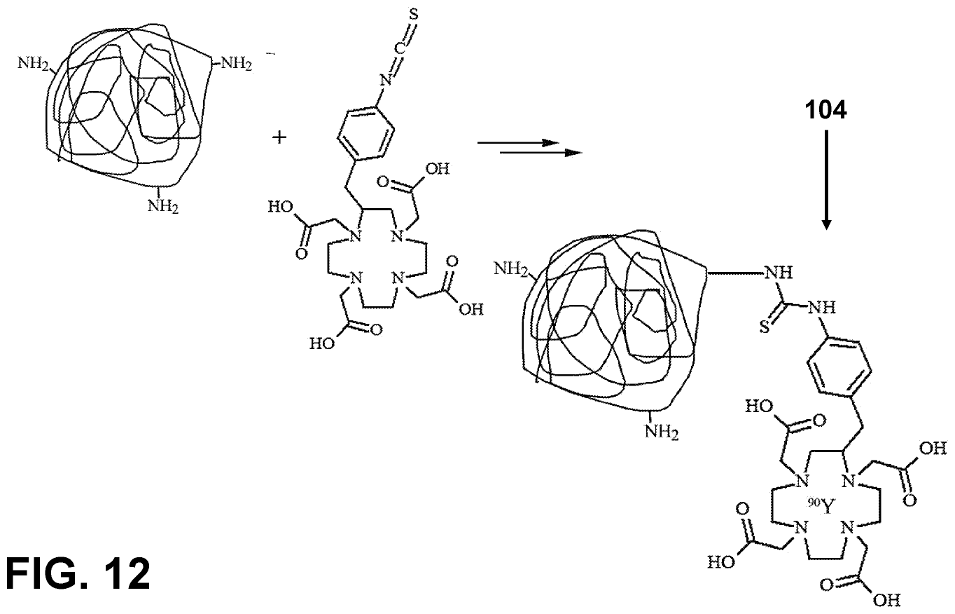

In another embodiment, as shown in FIG. 12, the second component 104 can include a radiolabeled peptide. The radiolabeled peptide can be crosslinked with the first component 102, which is not radioactive. In a specific, non-limiting example, the radiolabeled peptide can be formed from a precursor such as a dodecane tetraacetic acid based chelator and a protein. One skilled in the art can select other radiolabeled peptides and suitable cross-linkers to construct the radioactive biodegradable adhesive system 100.

In an additional example, the radioactive bolus 106 can include a tracking component. The tracking component can be a contrast agent such as an Mill recognizable compound or an iodinated CT contrast agent, as a non-limiting examples. In a more specific example, the MRI recognizable compound can be gadolinium. In another specific example, the CT contrast agent can include a 4-iodophenyl group. A skilled artisan can select other compounds to be used for the tracking component, within the scope of the present disclosure. Advantageously, by including the tracking component with the radioactive bolus 106, non-invasive post-treatment tracking can be completed more efficiently and more accurately. The location, shape, and maintenance of the bolus can therefore be noninvasively monitored. An embodiment of the polymerization reaction of the radioactive biodegradable adhesive system 100 is shown in reaction (2), where A represents the first component 102, B_{RAD} represents the second component 104, T represents the tracking component, and (A−B_{RAD}−T)ₓ represents x number of repeating polymerized units of the first component 102, the second component 104, and the tracking component:

A+B_{RAD}+T→(A−B_{RAD}−T)ₓ (2)

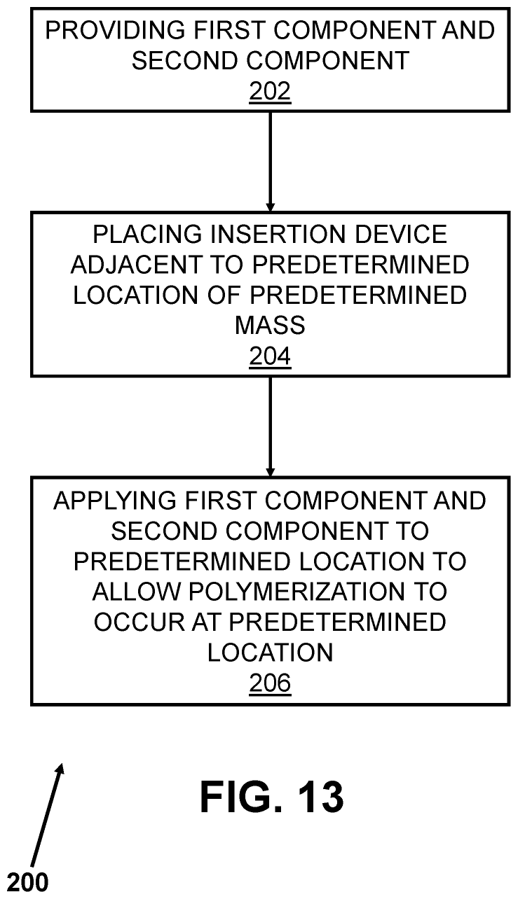

In a further embodiment, the present disclosure relates to a method 200 for treating a patient in need of brachytherapy with the radioactive biodegradable adhesive system 100, for example, as shown in FIGS. 3-7 and 13. The method 200 for constructing the radioactive biodegradable adhesive system 100 can include a first step 202 of providing the first component 102 and the second component 104. As described hereinabove, the second component 104 can include a radioactive material. The first component 102 and the second component 104 can be provided in the insertion device 110.

The method 200 can include a second step 204 of placing the insertion device 110 adjacent to the predetermined location of the predetermined mass 108 within the body 107 of the patient. The predetermined mass 108 can be a tumor. The predetermined location can include a biopsy site or a resection site of a tumor. A third step 206 of the method 200 can be applying the first component 102 and the second component 104 to the predetermined location to allow polymerization to occur at the predetermined location. The application can occur via the insertion device 110.

In one example, an operator can place the insertion tip 116 of the insertion device 110 into a bore hole after a biopsy procedure. Alternatively, the insertion tip 116 can include a syringe that can be inserted into the desired location with respect to the predetermined mass 108. The operator can then press the force applicator 118 to simultaneously dispense each of the first component 102 and the second component 104. When the first component 102 and the second component 104 meet at the predetermined location, the polymerization reaction can occur. Thus, the radioactive bolus 106 can be formed. The radioactive bolus 106 can fill the bore hole from the biopsy and the polymerization allows the radioactive bolus to be secured to the tumor. The radioactive bolus 106 can provide direct radiation from the radioactive material of the second component 104.

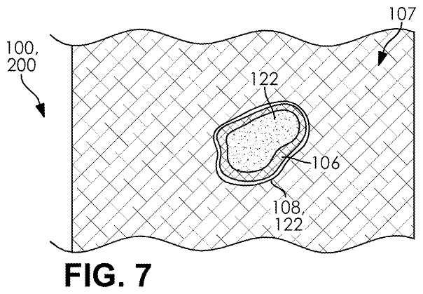

In another example, as shown in FIGS. 6-7, the operator can place the insertion tip 116 adjacent to a resected portion 122 of a tumor. The operator can then dispense the first component 102 and the second component 104 over the resected portion 122 of the tumor or predetermined mass 108. This process can be repeated as necessary over various locations on the surface of the predetermined mass 108, thereby, allowing multiple radioactive boluses 106 to form at multiple predetermined locations 108 to form, for example as shown in FIG. 5. In other examples, as shown in FIG. 6, an entire resected portion 122 can be brushed across a surface with a separate tool to form the radioactive bolus 106. Advantageously, where the radioactive bolus 106 is applied or brushed over a surface, this treatment can be used on a tissue surface after a predetermined mass has been resected. Desirably, without being bound to any particular theory, by applying or brushing the radioactive bolus 106 over the tissue surface after a predetermined mass has been resected, it is believed to militate against cancer recurrence. This form of treatment can be especially useful for surgeries involving bone metastasis, as a non-limiting example.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A radioactive biodegradable adhesive system, comprising:
a first component; and
a second component including a radioactive material;
wherein the first component and the second component are configured to polymerize to form a radioactive bolus.

2. The radioactive biodegradable adhesive system of claim 1, wherein the first component and the second component are provided in an insertion device.

3. The radioactive biodegradable adhesive system of claim 2, wherein the insertion device includes a first chamber, a second chamber, an insertion tip, and a force applicator, the first chamber containing the first component and the second chamber containing the second component.

4. The radioactive biodegradable adhesive system of claim 2, wherein the insertion device is configured to separately and simultaneously dispense the first component and the second component out of a distal end of the insertion tip.

5. The radioactive biodegradable adhesive system of claim 1, wherein the radioactive material includes one of an alpha emitter and a beta emitter.

6. The radioactive biodegradable adhesive system of claim 1, wherein the radioactive material includes yttrium-90.

7. The radioactive biodegradable adhesive system of claim 1, wherein the first component is a monomer and the second component is a radioactive catalyst.

8. The radioactive biodegradable adhesive system of claim 7, wherein the radioactive catalyst includes a dodecane tetraacetic acid derivative.

9. The radioactive biodegradable adhesive system of claim 1, wherein the first component includes a biopolymer and the second component includes another biopolymer.

10. The radioactive biodegradable adhesive system of claim 9, wherein the first component comprises a non-radioactive biopolymer crosslinker and the second component comprises a radioactive biopolymer crosslinker.

11. The radioactive biodegradable adhesive system of claim 1, wherein the first component includes a protein.

12. The radioactive biodegradable adhesive system of claim 11, wherein the protein includes reactive amine groups.

13. The radioactive biodegradable adhesive system of claim 1, wherein the second component includes an aldehyde.

14. The radioactive biodegradable adhesive system of claim 1, further comprising a contrast agent.

15. The radioactive biodegradable adhesive system of claim 14, wherein the contrast agent includes gadolinium.

16. The radioactive biodegradable adhesive system of claim 1, wherein the first component includes polylactate and an ethylene vinyl alcohol copolymer.

17. The radioactive biodegradable adhesive system of claim 1, wherein the first component includes chitosan.

18. A method for treating a predetermined location of a patient in need of brachytherapy, comprising:
providing a radioactive biodegradable adhesive system including a first component and a second component, the second component including a radioactive material, wherein the first component and the second component are configured to polymerize to form a radioactive bolus; and
applying the first component and the second component to the predetermined location to form the radioactive bolus at the predetermined location.

19. The method of claim 18, wherein the first component and the second component are provided in an insertion device.

20. The method of claim 19, wherein the insertion device includes a first chamber, a second chamber, an insertion tip, and a force applicator, the first chamber containing the first component and the second chamber containing the second component.
